(19) United States
(12) Patent Application Publication
(10) Pub. No.: US 2025/0164507 A1
(43) Pub. Date: May 22, 2025

(54) COMPOSITIONS AND METHODS FOR DIAGNOSING AND TREATING CARDIOMYOPATHY IN A FELINE

(71) Applicant: Société des Produits Nestlé S.A., vevey (CH)

(72) Inventor: Qinghong LI, Chesterfield, MO (US)

(73) Assignee: Société des Produits Nestlé S.A., vevey (CH)

(21) Appl. No.: 18/896,071

(22) Filed: Sep. 25, 2024

Related U.S. Application Data
(60) Provisional application No. 63/600,187, filed on Nov. 17, 2023.

(51) Int. Cl. G01N 33/68; G01N 33/74; G01N 33/92

(52) U.S. Cl. CPC G01N 33/6893; G01N 33/74; G01N 33/92; G01N 2333/575; G01N 2800/325; G01N 2800/52

(57) ABSTRACT

The present invention relates to methods and compositions for diagnosing and enabling treatment of cardiomyopathy (CM) in a feline. In one embodiment, a method of diagnosing CM in a feline can comprise measuring an amount of at least one metabolite from the feline; comparing the amount of the at least one metabolite from the feline to a corresponding predetermined value or a corresponding predetermined range; and diagnosing the feline as preclinical CM, clinical CM, or not having CM, based on the comparison.

### CROSS REFERENCE TO RELATED APPLICATION

This application claims the benefit of and priority to U.S. Provisional Application Ser. No. 63/600,187 filed Nov. 17, 2023, the disclosure of which is incorporated in its entirety herein by this reference.

### BACKGROUND

Cardiomyopathy (CM) is defined as a myocardial disorder in which the heart muscle is structurally and functionally abnormal in the absence of any other cardiovascular disease sufficient to cause the observed myocardial abnormality. Hypertrophic cardiomyopathy (HCM) is the most common type of cardiomyopathie phenotype, affecting 10-15% the general cat population. It is characterized with increased myocardial thickening associated with hypertrophied non-dilated left ventricle. The prevalence of HCM increases with age, with approximately 30% cats aged 9 years or older affected. Recent consensus statements of American College of Veterinary Internal Medicine (ACVIM) provided up-to-date guidelines for classification and diagnosis of cardiomyopathy in cats. Cats in stage A are predisposed to cardiomyopathy with no evidence of myocardial disease. Stage B refers to subclinical cats, which have cardiomyopathy, but without clinical signs. Stage B is further divided into two substages by means of atrial size, B1 with lower risk of imminent congestive heart failure (CHF) or arterial thromboembolism (ATE), while B2 with higher risk of imminent CHF or ATE. Cats with history of CHF or ATE are classified as stage C. The majority of the cats with HCM remain subclinical with prolonged stage B, while 30% developed clinical signs of CHF or ATE or both. Echocardiography is the gold standard for diagnosis of CMs in cats. However, the technology requires highly trained individuals with substantial experience. Heart murmurs are relatively common in adult cats, not every cat with a murmur has underlying heart disease. Other means, such as the electrocardiogra radiography are relatively insensitive for detection of heart enlargement in subclinical heart disease and the NT-proBNP assay does not diagnose heart disease.

Thus, there is a need for compositions and methods for diagnosing, treating, and preventing CMs in felines, especially diagnosing preclinical CMs. There is also a need for compositions and methods for treating CMs which provide partial or complete relief.

### SUMMARY

The present disclosure relates generally to compositions and methods for diagnosing and treatment of cardiomyopathy (CM) in a feline.

In one embodiment, a method of diagnosing CM in a feline can comprise measuring an amount of at least one metabolite from the feline, wherein the at least one metabolite is selected from the group consisting of tauro-beta-muricholate, 1-stearoyl-2-linoleoyl-GPE (18:0/18:2), 1-stearoyl-2-oleoyl-GPE (18:0/18:1), 1-stearoyl-2-linoleoyl-GPI (18:0/18:2), 1-behenoyl-GPC (22:0), 1-linoleoyl-GPI (18:2), taurodeoxycholic acid 3-sulfate, 1-palmitoyl-2-linoleoyl-GPE (16:0/18:2), asparagine, 1-palmitoyl-2-oleoyl-GPE (16:0/18:1), 1-arachidoyl-GPC (20:0), phosphatidylcholine (18:0/20:2, 20:0/18:2), taurodeoxycholate, glycerol, cystine, C-glycosyltryptophan, cysteine-glutathione disulfide, N-acetylglucosaminylasparagine, N-lactoyl valine, 5-(galactosylhydroxy)-lysine, 3-hydroxymyristate, hydroxyasparagine, guanidinosuccinate, N-lactoyl tyrosine, 1-methylhistidine, glucuronate, ribonate, 1-methylguanidine, N,N-dimethylalanine, 11-dehydrocorticosterone, cortisol, 2R,3R-dihydroxybutyrate, mannitol/sorbitol, n-propanol glucuronide, and mixtures thereof; comparing the amount of the at least one metabolite from the feline to a corresponding predetermined value or a corresponding predetermined range; and diagnosing the feline as preclinical CM, clinical CM, or not having CM, based on the comparison. In one aspect, the method of diagnosing can include utilizing the biomarkers and ranges found in Table 1.

In another embodiment, a method of enabling treatment or slowing progression of CM in a feline can comprise diagnosing preclinical CM or clinical CM in the feline and recommending a composition for the feline, wherein the composition treats or slows the progression of preclinical or clinical CM in the feline.

Additional features and advantages are described herein and will be apparent from the following Detailed Description.

### DETAILED DESCRIPTION

### Definitions

As used in this disclosure and the appended claims, the singular forms “a,” “an” and “the” include plural referents unless the context clearly dictates otherwise. Thus, for example, reference to “a composition” or “the composition” includes two or more compositions. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative, and are not exclusive or comprehensive.

As used herein, “about” is understood to refer to numbers in a range of numerals, for example the range of −10% to +10% of the referenced number, within −5% to +5% of the referenced number, or in one aspect, within −1% to +1% of the referenced number, and in a specific aspect, within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed. When reference is made to the pH, values correspond to pH measured at 25° C. with standard equipment. An “amount” can be the total amount of the referenced component per serving of the composition or per distinct unit of the composition and/or can be the weight percentage of the referenced component by dry weight. Moreover, an “amount” includes zero; for example, the recitation of an amount of a compound does not necessarily mean that the compound is present, unless followed by a range that excludes zero.

The terms “food,” “food product” and “food composition” mean a product or composition that is intended for ingestion by an animal and provides at least one nutrient to the animal. Further in this regard, these terms mean that the product or composition is in a form ready for consumption and is not merely an intermediate from which a consumable product or composition is made, although other food compositions can be added in some embodiments. The term “pet food” means any food composition intended to be consumed by a pet. The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal.

The term “animal” means any animal that has a need for the health benefits disclosed herein, including mammals, such as human, avian, bovine, canine, equine, feline, hircine, lupine, murine, ovine, or porcine animals.

The term “companion animal” means a dog or a cat. As used herein, the term “cat” and “feline” can be used interchangeably. In one embodiment, the companion animal can be a cat.

The term “complete and balanced” when referring to a food composition means a food composition that contains all known required nutrients in appropriate amounts and proportions based on recommendations of recognized authorities in the field of animal nutrition, and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO) as of Jan. 1, 2023.

As used herein, “biomarker” and “metabolite” can be used interchangeably and refer to a molecule that has been associated with CM in a feline.

As used herein, “cardiomyopathy” or “CM” is defined as the presence of structural or functional abnormalities of the heart muscle and includes hypertrophic cardiomyopathy (HCM), restrictive cardiomyopathy (RCM), nonspecific or unclassified cardiomyopathy (NCM), arrhythmogenic right ventricular cardiomyopathy (ARVC), and dilated cardiomyopathy (DCM).

As used herein, “hypertrophic cardiomyopathy” or “HCM” refers to a CM where the heart muscle in the left ventricle (the part of the heart that pumps blood out to the body) becomes thick (hypertrophied), reducing the amount of blood that can enter the ventricle. This decreases the heart's ability to supply blood and oxygen to the body and causes another part of the heart (left atrium) to dilate with the blood that can't flow normally into the stiffened, thickened ventricle.

As used herein, “restrictive cardiomyopathy” or “RCM” refers to a CM where scar tissue causes the heart muscles to stiffen, but unlike HCM, there is little or no muscle thickening (hypertrophy). The heart's ability to pump blood and oxygen to the body is impaired similarly to HCM.

As used herein, “nonspecific or unclassified cardiomyopathy” or “NCM” refers to a CM where the heart muscle has changes that cause a decrease in heart function but don't fit into the other CM categories. This condition could represent a combination of cardiomyopathies or a transition from one type to another.

As used herein, “arrhythmogenic right ventricular cardiomyopathy” or “ARVC” refers to a CM where the right ventricle (all or part of it) becomes very thin and is often replaced by scar tissue or fatty scar tissue. This results in a reduction of blood moving from the right ventricle into the lungs, backing up blood into the vessels of the abdomen.

As used herein, “dilated cardiomyopathy” or “DCM” refers to a CM where the heart muscle weakens and thins, losing the ability to pump blood to the body.

As used herein, “preclinical cardiomyopathy” or “preclinical CM” refers to stage A and/or stage B of CM.

As used herein, “clinical cardiomyopathy” or “clinical CM” refers to stage C and/or stage D of CM.

As used herein, “stage A” includes cats that are predisposed to cardiomyopathy but have no evidence of myocardial disease.

As used herein, “stage B” describes cats with cardiomyopathy but without clinical signs. Stage B is further divided into stage B1: cats at low risk of imminent congestive heart failure (CHF) or arterial thromboembolism (ATE), and stage B2: cats at higher risk of imminent CHF or ATE. Atrial size is an important prognostic marker, and it can be used as a means of subdividing cats with subclinical cardiomyopathy into low risk (B1) and higher risk (B2) cats. The more severe the left atrial (LA) enlargement, the higher the risk of CHF and ATE. Other factors that can taken into consideration when staging cats with subclinical cardiomyopathy can include LA and left ventricle (LV) systolic function and extreme LV hypertrophy.

As used herein, “stage C” refers to cats that have developed signs of CHF or ATE, even if clinical signs resolve with treatment.

As used herein, “stage D” refers to cats with signs of CHF refractory to treatment.

As used herein, “normalized relative abundance” refers to the area-under-the-curve of ion counts of each biomarker, as measured by liquid chromatography and mass spectrometry, further transformed using logarithm base 2 and auto-scaled to achieve a zero mean and unit variance. Such measurements are known in the art and have been described by Metabolon Inc. For example, details and further information are found in Ford L, Kennedy AD, Goodman KD et al. Precision of a Clinical Metabolomics Profiling Platform for Use in the Identification of Inborn Errors of Metabolism. J Appl Lab Med 2020;5:342-356 and Li Q, Larouche-Lebel E, Loughran KA, Huh TP, Suchodolski JS, Oyama MA. Metabolomics Analysis Reveals Deranged Energy Metabolism and Amino Acid Metabolic Reprogramming in Dogs With Myxomatous Mitral Valve Disease. J Am Heart Assoc 2021;10:e018923.

The term “B vitamin” refers to any B vitamin including derivatives, acidic forms, and salts thereof. Such B vitamins can include without limitation, vitamin B1 (thiamine), vitamin B2 (riboflavin), vitamin B3 (niacin, nicotinic acid, nicotinamide), vitamin B5 (pantothenic acid), vitamin B6 (pyridoxine, pyridoxal, pyridoxamine), vitamin B7 (biotin), vitamin B8 (myo-inositol), vitamin B9 (folic acid) vitamin B12 (cobalamin compounds including methylcobalamin, hydroxocobalamin, and cyanocobalamin).

“Wet food” means a pet food having a moisture content from about 50% to about 90%, and in one aspect, from about 70% to about 90%. “Dry food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%.

“Kibbles” is used synonymously with “chunks” herein and both terms mean pieces of dry or semi-moist pet food which can have a pellet shape or any other shape and can be made by slicing a food composition into separate pieces. Non-limiting examples of kibbles include particulates; pellets; pieces of pet food, dehydrated meat, meat analog, vegetables, and combinations thereof; and pet snacks, such as meat or vegetable jerky, rawhide, and biscuits. A “meat analog” is a meat emulsion product that resembles pieces of natural meat in appearance, texture, and physical structure.

A “medium chain triglyceride” is a lipid in which three fatty acids are bound by ester linkages to a glycerol backbone, and at least two and preferably all three of the fatty acids are each between six and twelve carbons in length. The medium-chain fatty acids are caproic acid (comprising six carbon atoms or C6:0), caprylic acid (comprising eight carbon atoms or C8:0), capric acid (comprising ten carbon atoms or C10:0) and lauric acid (comprising twelve carbon atoms or C12:0). In one embodiment, the medium-chain fatty acids are mainly (e.g., at least 98%) in the form of triglycerides. A composition comprising “lipids consisting essentially of medium chain triglycerides” contains medium chain triglycerides as at least 20% of the lipids, in some embodiments at least 30% of the lipids, in other embodiments at least 40% of the lipids, and in some embodiments at least 50% of the lipids in the composition.

The term “effective amount” of “therapeutically effect amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In some aspects, the particular disease, condition, or disorder can be cardio myopathy.

The term “dietary supplement” means a product that is intended to be ingested in addition to the normal animal diet. Dietary supplements may be in any form, e.g., solid, liquid, gel, tablets, capsules, powder, and the like. In one aspect, they can be provided in convenient dosage forms. In some embodiments, they can be provided in bulk consumer packages such as bulk powders, liquids, gels, or oils. In other embodiments, supplements can be provided in bulk quantities to be included in other food items such as snacks, treats, supplement bars, beverages and the like.

The term “long-term administration” means periods of repeated administration or consumption in excess of one month. Periods of longer than two, three, or four months can be used for certain embodiments. Also, more extended periods can be used that include longer than 5, 6, 7, 8, 9, or 10 months. Periods in excess of 11 months or 1 year can also be used. Longer term use extending over 1, 2, 3, or more years are included in the invention. For certain aging animals, the animal will continue consuming on a regular basis for the remainder of its life. This can also be referred to as consumption for “extended” periods.

The term “regular basis” means at least monthly dosing with the compositions or consumption of the compositions, and in one aspect, means at least weekly dosing. More frequent dosing or consumption, such as twice or three times weekly, can be performed in certain embodiments. Still, in other embodiments, regimens can be used that comprise at least once daily consumption.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Any embodiment disclosed herein can be combined with any other embodiment disclosed herein unless explicitly and directly stated otherwise.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

### Embodiments

In one embodiment, a method of diagnosing CM in a feline can comprise measuring an amount of at least one metabolite from the feline, wherein the at least one metabolite is selected from the group consisting of tauro-beta-muricholate, 1-stearoyl-2-linoleoyl-GPE (18:0/18:2), 1-stearoyl-2-oleoyl-GPE (18: 0/18:1), 1-stearoyl-2-linoleoyl-GPI (18:0/18:2), 1-behenoyl-GPC (22:0), 1-linoleoyl-GPI (18:2), taurodeoxycholic acid 3-sulfate, 1-palmitoyl-2-linoleoyl-GPE (16:0/18:2), asparagine, 1-palmitoyl-2-oleoyl-GPE (16:0/18:1), 1-arachidoyl-GPC (20:0), phosphatidylcholine (18:0/20:2, 20:0/18:2), taurodeoxycholate, glycerol, cystine, C-glycosyltryptophan, cysteine-glutathione disulfide, N-acetylglucosaminylasparagine, N-lactoyl valine, 5-(galactosylhydroxy)-lysine, 3-hydroxymyristate, hydroxyasparagine, guanidinosuccinate, N-lactoyl tyrosine, 1-methylhistidine, glucuronate, ribonate, 1-methylguanidine, N,N-dimethylalanine, 11-dehydrocorticosterone, cortisol, 2R,3R-dihydroxybutyrate, mannitol/sorbitol, n-propanol glucuronide, and mixtures thereof; comparing the amount of the at least one metabolite from the feline to a corresponding predetermined value or a corresponding predetermined range; and diagnosing the feline as preclinical CM, clinical CM, or not having CM, based on the comparison. In one aspect, the method of diagnosing can include utilizing the biomarkers found in Table 1, where the corresponding predetermined value or the corresponding predetermined range is from Table 1

In another embodiment, a method of enabling treatment or slowing progression of CM in a feline can comprise diagnosing preclinical CM or clinical CM in the feline and recommending a composition for the feline, wherein the composition treats or slows the progression of preclinical or clinical CM in the feline.

The present methods can further comprise obtaining a sample of at least one of serum or plasma from the feline, where the measuring of the amount of the metabolite(s) is performed on the sample of the serum or plasma.

Generally, the present methods of diagnosing a feline with a CM involve measuring the normalized relative abundance of a metabolite and determining that the feline has CM, including, in some aspects, whether the feline has preclinical CM or clinical CM, based on specific ranges as disclosed herein. Such diagnosis is possible based on the novel biomarkers and ranges discovered by the present inventors.

Generally, the present markers have unique ranges allowing for diagnosis between preclinical CM or clinical CM as shown in Table 1. Even so, the present methods can include the use of multiple markers to allow a diagnosis between preclinical CM or clinical CM. As such, in one embodiment, the method can further comprise measuring a normalized relative abundance of a second biomarker, a third biomarker, a fourth biomarker, a fifth biomarker, and/or a sixth biomarker, etc., and further determining that the feline has preclinical CM or clinical CM based on the majority of biomarkers.

To be clear, the present step of diagnosing can be based on one marker, two markers, three markers, four markers, five markers, six markers, seven markers, eight markers, nine markers, ten markers, and so on. When multiple markers are used, the diagnosis between preclinical and clinical can be based on a simple majority if the markers indicate more than one stage, e.g., if seven markers are used and five of the seven indicate preclinical CM and two of the seven indicate clinical CM, a diagnosis of preclinical can be made.

As such, in one aspect, the present methods can further comprise measuring at least two metabolites and diagnosing based on the comparison of each metabolite, wherein if each of the metabolites correspond to a different diagnosis, measuring an additional metabolite until a majority diagnosis is achieved.

In another aspect, the present methods can further comprise measuring at least three metabolites and diagnosing based on the comparison of each metabolite, wherein if each of the metabolites correspond to a different diagnosis, measuring an additional metabolite until a majority diagnosis is achieved.

In still another aspect, the method can further comprise measuring at least four metabolites and diagnosing based on the comparison of each metabolite, wherein if each of the metabolites correspond to a different diagnosis or a majority diagnosis is not achieved, measuring an additional metabolite until a majority diagnosis is achieved.

In yet another aspect, the method can further comprise measuring at least five metabolites and diagnosing based on the comparison of each metabolite, wherein if each of the metabolites correspond to a different diagnosis or a majority diagnosis is not achieved, measuring an additional metabolite until a majority diagnosis is achieved.

Generally, a method of enabling treatment, treating, or slowing progression of CM in a feline can comprise diagnosing CM in the feline as discussed herein and recommending or providing a composition for the feline, wherein the composition treats or slows the progression of CM in the feline.

Such compositions can comprise medium chain triglycerides and optionally one or more of omega-3 fatty acids, Vitamin E, Vitamin C, magnesium, or sulfur-containing amino acids (methionine, cysteine, taurine), glycine, glutamine, glutamate, tryptophan, bovine whey rich in cysteine-containing proteins and gamma-glutamyl-cysteine dipeptide. The composition can be a pet food, such as a wet pet food, a semi-moist pet food, or a dry pet food, e.g., kibble.

Generally, the medium chain triglycerides can be about 0.5 wt % to about 60 wt % of the composition. In one aspect, the medium chain triglycerides can be from about 1 wt % to about 20 wt % of the composition. In other aspects, the medium chain triglycerides can be from about 1 wt % to about 15 wt %, from about 1 wt % to about 10 wt %, or from about 2 wt % to about 10 wt % of the composition. The medium chain triglycerides may be prepared by any known process, such as direct esterification, rearrangement, fractionation and/or transesterification. For example, the medium chain triglycerides may be prepared from a source of vegetable oil, such as coconut oil, through a rearrangement process. The chain length and distribution thereof may vary depending on the source oil. For example, MCTs containing 1-10% C6, 30-60% C8, 30-60% C10 and 1-10% C12 can be derived from palm oil and/or coconut oil; in some embodiments, at least a portion of the MCTs are provided by coconut oil, but in other embodiments the composition does not contain coconut oil. MCTs containing at least about 95% C8 can be made by semi-synthetic esterification of octanoic acid to glycerin; in some embodiments thereof, the remainder of the fatty acids are C6 and C10. Mixtures comprising MCTs with about 50% total C8 and/or about 50% total C10 are also useful herein.

Non-limiting examples of suitable omega-3 fatty acids include eicosapentaenoic acid (EPA), docosahexaenoic acid (DHA), alpha-linolenic acid (ALA) and mixtures thereof. In one embodiment, the omega-3 fatty acids can range from about 0.2 wt % to about 3 wt % of the composition. In some embodiments, the omega-3 fatty acids are at least about 0.2 wt %, at least about 1.0 wt %, or at least about 2.0 wt %.

Non-limiting examples of suitable sulfur-containing amino acids include methionine, cysteine, taurine, and mixtures thereof.

The present methods can include identifying a feline as being at risk of CM prior to the administering of the composition. A non-limiting example of a characteristic that can identify a companion animal as being at risk of CM include an increased concentration of NT-proBNP.

Generally, such compositions can be recommended to the pet owner or can be administered to the animal. In some embodiments, the composition can be administered to the animal for a period of at least one week, at least one month, at least two, three, four, five or six months; and in some embodiments, for at least one year. During the period, the composition can be administered to the animal at least one day per week, at least two days per week, at least three, four, five or six days per week; or even seven days per week. The composition can be administered in a single dose per day or in multiple separate doses per day. In an embodiment, the composition can be administered in an amount that provides about 0.001 g to 50 g of the MCTs per kg body weight of the animal (e.g., a companion animal) per day. In one aspect, 0.1 g to about 5 g of the MCTs per kg body weight of the animal (e.g., a companion animal) can be administered per day.

In an embodiment, the composition further comprises (i) carnitine, (ii) glycine and methionine, (iii) glutamine, (iv) an antioxidant such as vitamin E and vitamin C, or (v) mixtures thereof. The composition can be high in protein, for example at least about 20 wt %, at least about 25 wt %, or even at least about 30 wt % of the composition. Additionally, the composition can have balanced amounts of magnesium, sodium and potassium; for example, the ratio of potassium to sodium can be about 4:1 to about 1:1, in one aspect, about 4:1 to about 2:1, with the magnesium in an amount of about 0.08 wt % to about 0.25 wt %, and in one aspect, from about 0.10 wt % to about 0.15 wt %. At least a portion of the magnesium, sodium and potassium can be provided as isolated compounds (e.g., salts). Alternatively or additionally, at least a portion of the magnesium, sodium and potassium can be provided by one or more foodstuffs. For example, magnesium can be provided by wheat bran, whole grains, leafy green vegetables, meat, beans and bananas; and potassium and sodium can be provided by meats, fish, whole grains, yogurt, bananas, sweet potatoes, squash, beans and tomatoes.

The food compositions disclosed herein can be any food formulated for consumption by an animal. In one embodiment, the food compositions can be pet food compositions. In one aspect, the pet food compositions can be formulated for a feline. In an embodiment, the pet food composition provides complete nutrition as defined by the Association of American Feed Control Officials (AAFCO) and which depends on the type of animal for which the composition is intended (e.g., a cat).

In one embodiment, the food compositions can comprise protein in amounts from about 10%, 20%, 30%, 35%, 40%, 45%, 50%, or even 55% to about 20%, 25%, 30%, 35%, 40%, 45%, 50%, 55%, 60%, or even 70% by weight, including various subranges within these amounts. In one aspect, the protein can be from about 20% to about 60% of the food composition by weight. In another aspect, the protein can be from about 25% to about 50% of the food composition by weight.

The food composition can comprise meat, such as emulsified meat. Examples of suitable meat include poultry, beef, pork, lamb and fish, especially those types of meats suitable for pets. The meat can include any additional parts of an animal including offal. Some or all of the meat can be provided as one or more meat meals, namely meat that has been dried and ground to form substantially uniform-sized particles and as defined by AAFCO. Additionally or alternatively, vegetable protein can be used, such as pea protein, corn protein (e.g., ground corn or corn gluten), wheat protein (e.g., ground wheat or wheat gluten), soy protein (e.g., soybean meal, soy concentrate, or soy isolate), rice protein (e.g., ground rice or rice gluten) and the like.

In one embodiment, the food compositions can comprise fat in amounts from about 5%, 10%, 15%, 20%, 25%, 30%, 35%, 40%, 45%, or even 50% to about 15%, 20%, 25%, 30%, 35%, 40%, 45%, 50%, 55%, or even 60%, including various subranges within these amounts by weight. In one aspect, the fat comprises from about 10% to about 40% of the food composition by weight. In another aspect, the fat comprises from about 20% to about 35% of the food composition by weight.

The food compositions disclosed herein can comprise one or more of a vegetable oil, a flavorant, a colorant or water. Non-limiting examples of suitable vegetable oils include soybean oil, corn oil, cottonseed oil, sunflower oil, canola oil, peanut oil, safflower oil and the like. In some embodiments, the lipids in the composition can consist of the MCTs and one or more of any vegetable oil, any fish oil, the lipid from any meat, and any omega-3 fatty acids.

Non-limiting examples of suitable flavorants include yeast, tallow, rendered animal meals (e.g., poultry, beef, lamb, pork), flavor extracts or blends (e.g., grilled beef), animal digests, and the like. Non-limiting examples of suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The food compositions disclosed herein can optionally include additional ingredients, such as starches, humectants, oral care ingredients, preservatives, amino acids, fibers, prebiotics, sugars, animal oils, aromas, other oils additionally or alternatively to vegetable oil, salts, vitamins, minerals, probiotic microorganisms, bioactive molecules or combinations thereof.

In one embodiment, the carbohydrate comprises from about 1% to about 70% of the food composition by weight. In another embodiment, the carbohydrate comprises from about 20% to about 60% of the food compositions by weight. In other aspects, the carbohydrate can be present in amounts from about 1%, 5%, 10%, 20%, 30%, 40%, or even 50%, to about 5%, 10%, 20%, 30%, 40%, 50%, 60%, or even 70% by weight.

Non-limiting examples of suitable starches include a grain such as corn, rice, wheat, barley, oats, potatoes, peas, beans, cassava, and the like, and mixtures of these grains, and can be included at least partially in any flour. Non-limiting examples of suitable humectants include salt, sugars, propylene glycol and polyhydric glycols such as glycerin and sorbitol, and the like. Non-limiting examples of suitable oral care ingredients include alfalfa nutrient concentrate containing chlorophyll, sodium bicarbonate, phosphates (e.g., tricalcium phosphate, acid pyrophosphates, tetrasodium pyrophosphate, metaphosphates, and orthophosphates), peppermint, cloves, parsley, ginger and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

Yet another aspect of the present disclosure is a method of making a pet food, the method comprising combining any of the comestible ingredients described above, mixing the ingredients, extruding the mixture, optionally, drying the extrudate, and optionally cutting or breaking the extrudate into individual kibbles.

Other compositions that can be beneficial for providing nutrition to felines having CM can include protein, carbohydrates, fat, and fiber. In another embodiment, the composition can contain less than 1% of phosphorous compounds and/or phosphate compounds. In still another embodiment, the composition can contain less than 1% of potassium. In yet another embodiment, the composition can have protein and phosphorus in a ratio between 5:1 and 15:1. In one embodiment, the composition can comprise arginine, eicosapentaenoic acid, docosahexaenoic acid, vitamin E, and B vitamins. Such B vitamins can include any combination of vitamin B1 (thiamine), vitamin B2 (riboflavin), vitamin B3 (niacin, nicotinic acid, nicotinamide), vitamin B5 (pantothenic acid), vitamin B6 (pyridoxine, pyridoxal, pyridoxamine), vitamin B7 (biotin), vitamin B8 (myo-inositol), vitamin B9 (folic acid) vitamin B12 (cobalamin compounds including methylcobalamin, hydroxocobalamin, and cyanocobalamin).

Generally, the composition can be administered sufficiently such that the treatment is effective. In one aspect, the administration can be on a regular basis. In another aspect, the administration can be a long-term administration. Administration of the composition can include any manner of delivery. In one embodiment, the composition can be administered in conjunction with a pet food composition. In another embodiment, the composition is a pet food. In still another embodiment, the composition can be a sachet or supplement administered in conjunction with a pet food. In yet another embodiment, the composition can be a sachet or supplement administered separately from other food compositions.

Additionally, the present compositions can comprise omega-3 fatty acids. Non-limiting examples of suitable omega-3 fatty acids include eicosapentaenoic acid (EPA), docosahexaenoic acid (DHA), alpha-linolenic acid (ALA) and mixtures thereof. In one embodiment, the omega-3 fatty acids can range from about 0.2%, 0.5%, 1%, 2%, or even 3% to about 1%, 2%, 3%, 4%, or even 5% of the composition by weight. In some embodiments, the omega-3 fatty acids are present in the food composition in an amount from about 1% to about 5% by weight. In some embodiments, the omega-3 fatty acids are present in the food composition in an amount from about 1% to about 2% by weight.

In addition to the fats and fatty acids discussed herein, the present compositions can comprise omega-6 fatty acids. Non-limiting examples of suitable omega-6 fatty acids include linoleic acid, gamma-linolenic acid (GLA), arachidonic acid (AA, ARA), eicosadienoic acid, docosadienoic acid, and mixtures thereof. In one embodiment, the omega-6 fatty acids can range from about 0.2%, 0.5%, 1%, 2%, or even 3% to about 1%, 2%, 3%, 4%, or even 5% of the composition by weight. In some embodiments, the omega-6 fatty acids are present in the food composition in an amount from about 1% to about 5% by weight. In some embodiments, the omega-6 fatty acids are present in the food composition in an amount from about 1% to about 2% by weight.

According to the presently described methods, administration, including administration as part of a dietary regimen, can span a period ranging from parturition through the adult life of the animal. In certain embodiments, the animal can be a young or growing animal. In other embodiments, administration can begin, for example, on a regular or extended regular basis, when the animal has reached more than about 10%, 20%, 30%, 40%, or 50% of its projected or anticipated lifespan. In some embodiments, the animal can have attained 40, 45, or 50% of its anticipated lifespan. In yet other embodiments, the animal can be older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

Such administration can be performed for a time required to accomplish one or more objectives described herein, e.g., treating CM. Other administration amounts may be appropriate and can be determined based on the animal's initial weight as well as other variables such as species, gender, breed, age, desired health benefit, etc.

The moisture content for pet food compositions varies depending on the nature of the food composition. The food compositions may be dry compositions (e.g., kibble), semi-moist compositions, wet compositions, or any mixture thereof. In one embodiment, the composition can be a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one embodiment, the compositions can have moisture content from about 5% to about 20%. Dry food products include a variety of foods of various moisture contents, such that they are relatively shelf-stable and resistant to microbial or fungal deterioration or contamination. Also, in one aspect, dry food compositions can be extruded food products for companion animals.

The food compositions may also comprise one or more fiber sources. Such fiber sources include fiber that is soluble, insoluble, fermentable, and nonfermentable. Such fibers can be from plant sources such as marine plants, but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, corn bran, wheat bran, oat bran, carob bean gum, citrus pulp, pectin, fructooligosaccharide, short chain oligofructose, mannanoligofructose, soy fiber, arabinogalactan, galactooligosaccharide, arabinoxylan, cellulose, chicory, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefits described herein or to the immune system of an animal.

In one embodiment, the food compositions can include a total dietary fiber from about 1% to about 15% by weight. In some aspects, the total dietary fiber can be included in an amount from about 5% to about 15% by weight, or even from about 8% to about 13% by weight. In another embodiment, the food compositions can include crude fiber from about 1% to about 10% by weight. In some aspects, the crude fiber can be included in an amount from about 3% to about 10% by weight, or even from about 3% to about 7% by weight.

In some embodiments, the ash content of the food composition ranges from less than 1% to about 15%. In one aspect, the ash content can be from about 5% to about 10%.

Generally, the food composition can be a meal, component of a meal, a snack, supplement, or a treat. Such compositions can include complete foods intended to supply the necessary dietary requirements for an animal.

As discussed herein, pet food compositions may further comprise one or more substances such as vitamins, minerals, antioxidants, probiotics, prebiotics, salts, and functional additives such as palatants, colorants, emulsifiers, and antimicrobial or other preservatives. Non-limiting examples of suitable preservatives include potassium sorbate, sorbic acid, sodium methyl para-hydroxybenzoate, calcium propionate, propionic acid, and combinations thereof. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat-soluble vitamins as A, D, E, and K and water-soluble vitamins including B vitamins, and vitamin C. Inulin, amino acids, enzymes, coenzymes, and the like may be useful to include in various embodiments.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

For example, the amount of any of the above-noted ingredients can be decreased or increased based on the estimated effect on CM in a feline.

### EXAMPLES

The following non-limiting examples are illustrative of embodiments of the present disclosure.

### Example 1—Cardiomyopathy (CM) Study of Felines

Twenty-three healthy cats (ACVIM stage A) and 60 cats with various severity of CMs were enrolled. Among them, 31 and 12 cats had stages B1 and B2 preclinical CM respectively, while 17 cats had history of congestive heart failure (CHF) also known as stage C or clinical CM. Venous blood sample of 2 to 3 mL was collected from each cat and allowed to clot and centrifuged at 1600 g for 5 minutes to yield serum samples, which were stored at −80°° C. until use.

Untargeted metabolomics assays were performed at a commercial laboratory (Metabolon, Inc.). Sample preparation and extraction, liquid chromatography, and mass spectrometry followed Metabolon standard protocols as described in Ford L, Kennedy AD, Goodman KD et al. Precision of a Clinical Metabolomics Profiling Platform for Use in the Identification of Inborn Errors of Metabolism. J Appl Lab Med 2020;5:342-356. Compound detection and identification were performed using Metabolon proprietary software and database. To identify differential metabolites, a multiple linear regression, adjusted for sex, age, and body weight was performed. P values were adjusted to control the false discovery rate (FDR) using the Benjamini-Hochberg method. Significant metabolites were subjected to pairwise comparisons using the R Package “pairwise.t.test” with pooled SD and Benjamini-Hochberg adjustment for multiple testing. Adjusted P value ≤0.05 was considered significant.

Normalized relative abundance was calculated for the serum metabolites with differentiation of preclinical CM cats, clinical CM cats, versus healthy cats as shown in Table 1.

TABLE 1

| | Diagnostic Range | | |
|---|---|---|---|
| | (measured as normalized | | |
| | relative abundance) | | |
| Chemical | Healthy | Preclinical | Clinical |
| tauro-beta-muricholate | >−0.682 | [−1.689, −0.682] | <−1.689 |
| (CAS 25696-60-0) | | | |
| 1-stearoyl-2-linoleoyl-GPE (18:0/18:2) | >0.042 | [−0.6927, 0.042] | <−0.6927 |
| (also referred to as 1-Stearoyl-2-linoleoyl-sn- | | | |
| glycero-3-phosphatidylcholine) | | | |
| (PubChem CID 6441487) | | | |
| 1-stearoyl-2-oleoyl-GPE (18:0/18:1) | >0.046 | [−0.8282, 0.046] | <−0.8282 |
| (also referred to as 1-octadecanoyl-2-(9Z- | | | |
| octadecenoyl)-sn-glycero-3-phosphoethanolamine) | | | |
| (PubChem CID 9546742) | | | |
| 1-stearoyl-2-linoleoyl-GPI (18:0/18:2) | >−0.009 | [−0.2701, −0.009] | <−0.2701 |
| (also referred to as (2R)-1-[(Hydroxy{[(1s,3R)- | | | |
| 2,3,4,5,6- | | | |
| pentahydroxycyclohexyl]oxy}phosphoryl)oxy]-3- | | | |
| (stearoyloxy)-2-propanyl (9Z,12Z)-9,12- | | | |
| octadecadienoate) | | | |
| (ChemSpider 24767694) | | | |
| 1-behenoyl-GPC (22:0) | >0.053 | [−0.0355, 0.053] | <−0.0355 |
| (also referred to as 1-Behenoyl-2-hydroxy-sn- | | | |
| glycero-3-phosphocholine) | | | |
| (PubChem CID 24779479) | | | |
| 1-linoleoyl-GPI (18:2) | >0.147 | [−0.0834, 0.147] | <−0.0834 |
| (also referred to as (2R)-2-hydroxy-3- | | | |
| [(hydroxy{[(1S,2R,3R,4S,5S,6R)-2,3,4,5,6- | | | |
| pentahydroxycyclohexyl]oxy}phosphoryl)oxy]propyl | | | |
| (9Z,12Z)-octadeca-9,12-dienoate) | | | |
| (PubChem CID 11124828) | | | |
| taurodeoxycholic acid 3-sulfate | >−0.578 | [−1.2202, −0.578] | <−1.2202 |
| (PubChem CID 162641067) | | | |
| 1-palmitoyl-2-linoleoyl-GPE (16:0/18:2) | >0.1 | [−0.6725, 0.1] | <−0.6725 |
| (also referred to as 1-hexadecanoyl-2-(9Z,12Z- | | | |
| octadecadienoyl)-sn-glycero-3- | | | |
| phosphoethanolamine) | | | |
| (PubChem CID 9546747) | | | |
| Asparagine | >0.076 | [−0.0313, 0.076] | <−0.0313 |
| (CAS 70-47-3) | | | |
| 1-palmitoyl-2-oleoyl-GPE (16:0/18:1) | >−0.032 | [−0.8361, −0.032] | <−0.8361 |
| (also referred to as 1-Palmitoyl-2-oleoyl-sn-glycero- | | | |
| 3-phosphoethanolamine) | | | |
| (PubChem CID 5283496) | | | |
| 1-arachidoyl-GPC (20:0) | >0.097 | [−0.0527, 0.097] | <−0.0527 |
| (also referred to as 1-Eicosanoyl-sn-glycero-3- | | | |
| phosphocholine) | | | |
| (PubChem CID 24779473) | | | |
| phosphatidylcholine (18:0/20:2, 20:0/18:2) | >0.01 | [−0.1297, 0.01] | <−0.1297 |
| (also referred to as (2-{[(2R)-2-(icosanoyloxy)-3- | | | |
| [(9Z,12Z)-octadeca-9,12-dienoyloxy]propyl | | | |
| phosphonato]oxy}ethyl)trimethylazanium and (2- | | | |
| {[(2R)-2-[(11Z,14Z)-icosa-11,14-dienoyloxy]-3- | | | |
| (octadecanoyloxy)propyl | | | |
| phosphonato]oxy}ethyl)trimethylazanium) | | | |
| (HMDB0008143, HMDB0008045) | | | |
| Taurodeoxycholate | >−0.568 | [−1.5714, −0.568] | <−1.5714 |
| (CAS 207737-97-1) | | | |
| Glycerol | <−0.139 | [−0.139, 0.3271] | >0.3271 |
| (CAS 56-81-5) | | | |
| Cystine | <−0.099 | [−0.099, 0.1832] | >0.1832 |
| (CAS 56-89-3) | | | |
| C-glycosyltryptophan | <−0.09 | [−0.09, −0.004] | >−0.004 |
| (CAS 180509-18-6) | | | |
| cysteine-glutathione disulfide | <−0.073 | [−0.073, 0.1492] | >0.1492 |
| (CAS 13081-14-6) | | | |
| N-acetylglucosaminylasparagine | <−0.293 | [−0.293, −0.027] | >−0.027 |
| (CAS 2776-93-4) | | | |
| N-lactoyl valine | <−0.184 | [−0.184, −0.0656] | >−0.0656 |
| (CAS 21753-44-6) | | | |
| 5-(galactosylhydroxy)-lysine | <−0.05 | [−0.05, −0.0732] | >−0.0732 |
| (CAS 32448-36-5) | | | |
| 3-hydroxymyristate | <−0.123 | [−0.123, 0.135] | >0.135 |
| (CAS 3422-31-9) | | | |
| Hydroxyasparagine | <−0.021 | [−0.021, 0.0491] | >0.0491 |
| (PubChem CID 15991574) | | | |
| Guanidinosuccinate | <−0.155 | [−0.155, 0.1489] | >0.1489 |
| (CAS 6133-30-8) | | | |
| N-lactoyl tyrosine | <−0.237 | [−0.237, −0.1281] | >−0.1281 |
| (CAS 1373771-50-6) | | | |
| 1-methylhistidine | <−0.048 | [−0.048, 0.0389] | >0.0389 |
| (CAS 332-80-9) | | | |
| Glucuronate | <−0.109 | [−0.109, 0.1462] | >0.1462 |
| (CAS 207300-70-7) | | | |
| Ribonate | <−0.051 | [−0.051, 0.1058] | >0.1058 |
| (CAS 5336-08-3) | | | |
| 1-methylguanidine | <−0.181 | [−0.181, −0.202] | >−0.202 |
| (CAS 22661-87-6) | | | |
| N,N-dimethylalanine | <−0.489 | [−0.489, −0.1014] | >−0.1014 |
| (CAS 2812-31-9) | | | |
| 11-dehydrocorticosterone | <−0.26 | [−0.26, 0.21] | >0.21 |
| (CAS 72-23-1) | | | |
| Cortisol | <−0.306 | [−0.306, 0.3537] | >0.3537 |
| (CAS 50-23-7) | | | |
| 2R,3R-dihydroxybutyrate | <−0.029 | [−0.029, 0.2423] | >0.2423 |
| (CAS 759-06-8) | | | |
| mannitol/sorbitol | <−0.027 | [−0.027, 0.6064] | >0.6064 |
| (PubChem CID 5780, 6251) | | | |
| n-propanol glucuronide | <−0.418 | [−0.418, 0.7998] | >0.7998 |
| (PubChem CID 119515) | | | |

Based on the metabolite biomarkers in Table 1, the present metabolite biomarkers can be used to diagnose CM in cats as well as specific diagnosis of preclinical CM and clinical CM. The brackets in Table 1 indicate a range of values identified by endpoints separated by a comma, with the endpoints included within the range. The present methods can include measuring a normalized relative abundance of the metabolite biomarker and determining that the feline has preclinical CM or clinical CM if the normalized relative abundance of the metabolite biomarker is in the range according to the preclinical CM or clinical CM columns, respectively, according to Table 1. Additionally, further diagnosis can be made if the multiple markers are used. For example, if two metabolite biomarkers are used and both correspond to the same diagnosis, e.g, preclinical CM, then the diagnosis would be preclinical CM. However, if one metabolite biomarker corresponds to preclinical CM and the other metabolite biomarker corresponds to clinical CM, an additional metabolite biomarker can be measured to achieve a majority diagnosis. As such, the present methods can also further comprise measuring additional metabolite biomarkers to determine if the feline has preclinical CM, clinical CM, or is healthy according to the ranges listed in Table 1.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of diagnosing cardiomyopathy (CM) in a feline, the method comprising: measuring an amount of at least one metabolite from the feline, wherein the at least one metabolite is selected from the group consisting of tauro-beta-muricholate, 1-stearoyl-2-linoleoyl-GPE (18:0/18:2), 1-stearoyl-2-oleoyl-GPE (18: 0/18:1), 1-stearoyl-2-linoleoyl-GPI (18:0/18:2), 1-behenoyl-GPC (22:0), 1-linoleoyl-GPI (18:2), taurodeoxycholic acid 3-sulfate, 1-palmitoyl-2-linoleoyl-GPE (16:0/18:2), asparagine, 1-palmitoyl-2-oleoyl-GPE (16:0/18:1), 1-arachidoyl-GPC (20:0), phosphatidylcholine (18:0/20:2, 20:0/18:2), taurodeoxycholate, glycerol, cystine, C-glycosyltryptophan, cysteine-glutathione disulfide, N-acetylglucosaminylasparagine, N-lactoyl valine, 5-(galactosylhydroxy)-lysine, 3-hydroxymyristate, hydroxyasparagine, guanidinosuccinate, N-lactoyl tyrosine, 1-methylhistidine, glucuronate, ribonate, 1-methylguanidine, N,N-dimethylalanine, 11-dehydrocorticosterone, cortisol, 2R,3R-dihydroxybutyrate, mannitol/sorbitol, n-propanol glucuronide, and mixtures thereof;
comparing the amount of the at least one metabolite from the feline to a corresponding predetermined value or a corresponding predetermined range; and
diagnosing the feline as preclinical CM, clinical CM, or not having CM, based on the comparison.

2. The method of claim 1, comprising obtaining a sample of at least one of serum or plasma from the feline, wherein the measuring of the amount of each of the at least one metabolite is performed on the sample of at least one of serum or plasma.

3. The method of claim 1, wherein the corresponding predetermined value or the corresponding predetermined range is from Table 1:
TABLE 1

| | Diagnostic Range | | |
|---|---|---|---|
| | (measured as normalized | | |
| | relative abundance) | | |
| Chemical | Healthy | Preclinical | Clinical |
| tauro-beta-muricholate | >−0.682 | [−1.689, −0.682] | <−1.689 |
| 1-stearoyl-2-linoleoyl-GPE (18:0/18:2) | >0.042 | [−0.6927, 0.042] | <−0.6927 |
| 1-stearoyl-2-oleoyl-GPE (18:0/18:1) | >0.046 | [−0.8282, 0.046] | <−0.8282 |
| 1-stearoyl-2-linoleoyl-GPI (18:0/18:2) | >−0.009 | [−0.2701, −0.009] | <−0.2701 |
| 1-behenoyl-GPC (22:0) | >0.053 | [−0.0355, 0.053] | <−0.0355 |
| 1-linoleoyl-GPI (18:2) | >0.147 | [−0.0834, 0.147] | <−0.0834 |
| taurodeoxycholic acid 3-sulfate | >−0.578 | [−1.2202, −0.578] | <−1.2202 |
| 1-palmitoyl-2-linoleoyl-GPE (16:0/18:2) | >0.1 | [−0.6725, 0.1] | <−0.6725 |
| Asparagine | >0.076 | [−0.0313, 0.076] | <−0.0313 |
| 1-palmitoyl-2-oleoyl-GPE (16:0/18:1) | >−0.032 | [−0.8361, −0.032] | <−0.8361 |
| 1-arachidoyl-GPC (20:0) | >0.097 | [−0.0527, 0.097] | <−0.0527 |
| phosphatidylcholine (18:0/20:2, 20:0/18:2) | >0.01 | [−0.1297, 0.01] | <−0.1297 |
| Taurodeoxycholate | >−0.568 | [−1.5714, −0.568] | <−1.5714 |
| Glycerol | <−0.139 | [−0.139, 0.3271] | >0.3271 |
| Cystine | <−0.099 | [−0.099, 0.1832] | >0.1832 |
| C-glycosyltryptophan | <−0.09 | [−0.09, −0.004] | >−0.004 |
| cysteine-glutathione disulfide | <−0.073 | [−0.073, 0.1492] | >0.1492 |
| N-acetylglucosaminylasparagine | <−0.293 | [−0.293, −0.027] | >−0.027 |
| N-lactoyl valine | <−0.184 | [−0.184, −0.0656] | >−0.0656 |
| 5-(galactosylhydroxy)-lysine | <−0.05 | [−0.05, −0.0732] | >−0.0732 |
| 3-hydroxymyristate | <−0.123 | [−0.123, 0.135] | >0.135 |
| Hydroxyasparagine | <−0.021 | [−0.021, 0.0491] | >0.0491 |
| Guanidinosuccinate | <−0.155 | [−0.155, 0.1489] | >0.1489 |
| N-lactoyl tyrosine | <−0.237 | [−0.237, −0.1281] | >−0.1281 |
| 1-methylhistidine | <−0.048 | [−0.048, 0.0389] | >0.0389 |
| Glucuronate | <−0.109 | [−0.109, 0.1462] | >0.1462 |
| Ribonate | <−0.051 | [−0.051, 0.1058] | >0.1058 |
| 1-methylguanidine | <−0.181 | [−0.181, −0.202] | >−0.202 |
| N,N-dimethylalanine | <−0.489 | [−0.489, −0.1014] | >−0.1014 |
| 11-dehydrocorticosterone | <−0.26 | [−0.26, 0.21] | >0.21 |
| Cortisol | <−0.306 | [−0.306, 0.3537] | >0.3537 |
| 2R,3R-dihydroxybutyrate | <−0.029 | [−0.029, 0.2423] | >0.2423 |
| mannitol/sorbitol | <−0.027 | [−0.027, 0.6064] | >0.6064 |
| n-propanol glucuronide | <−0.418 | [−0.418, 0.7998] | >0.7998 |

4. The method of claim 1, further comprising measuring at least two metabolites and diagnosing based on the comparison of each metabolite, wherein if each of the metabolites correspond to a different diagnosis, measuring an additional metabolite until a majority diagnosis is achieved.

5. The method of claim 1, further comprising measuring at least three metabolites and diagnosing based on the comparison of each metabolite, wherein if each of the metabolites correspond to a different diagnosis, measuring an additional metabolite until a majority diagnosis is achieved.

6. The method of claim 1, further comprising measuring at least four metabolites and diagnosing based on the comparison of each metabolite, wherein if each of the metabolites correspond to a different diagnosis or a majority diagnosis is not achieved, measuring an additional metabolite until a majority diagnosis is achieved.

7. The method of claim 1, further comprising measuring at least five metabolites and diagnosing based on the comparison of each metabolite, wherein if each of the metabolites correspond to a different diagnosis or a majority diagnosis is not achieved, measuring an additional metabolite until a majority diagnosis is achieved.

8. A method of enabling treatment or slowing progression of CM in a feline, comprising diagnosing preclinical CM or clinical CM in the feline according to claim 1 and recommending a composition for the feline, wherein the composition treats or slows the progression of preclinical or clinical CM in the feline.

9. The method of claim 8, wherein the composition has a characteristic selected from the group consisting of (i) ratio of potassium to sodium from about 4:1 to about 1:1, (ii) magnesium in an amount from about 0.08 wt % to about 0.25 wt %, (iii) protein in an amount of at least 20 wt %, and (iv) combinations thereof.

10. The method of claim 8, wherein the composition comprises medium chain triglycerides.

11. The method of claim 8, wherein the composition further comprises a component selected from the group consisting of an omega-3 fatty acid, Vitamin E, magnesium, glycine, a sulfur-containing amino acid, and mixtures thereof.

12. The method of claim 11, wherein the omega-3 fatty acid is selected from the group consisting of eicosapentaenoic acid (EPA), docosahexaenoic acid (DHA), and mixtures thereof.

13. The method of claim 11, wherein the sulfur-containing amino acid is selected from the group consisting of methionine, cysteine, taurine, and mixtures thereof.

14. The method of claim 8, wherein the composition is a complete and nutritionally balanced pet food.

15. The method of claim 8, wherein the composition further comprises (i) carnitine, (ii) glycine and methionine, (iii) glutamine, (iv) an antioxidant such as vitamin E and vitamin C, or (v) mixtures thereof.
